(19) United States
(12) Patent Application Publication
(10) Pub. No.: US 2013/0182222 A1
(43) Pub. Date: Jul. 18, 2013

(54) SYSTEMS AND METHODS FOR OCULAR FUNDUS EXAMINATION REFLECTION REDUCTION

(71) Applicants: Paul Andrew Yates, Charlottesville, VA (US); Thomas A. Mendel, Charlottesville, VA (US); Kenneth Tran, Burke, VA (US)

(72) Inventors: Paul Andrew Yates, Charlottesville, VA (US); Thomas A. Mendel, Charlottesville, VA (US); Kenneth Tran, Burke, VA (US)

(73) Assignee: UNIVERSITY OF VIRGINIA PATENT FOUNDATION, CHARLOTTESVILLE, VA (US)

(21) Appl. No.: 13/699,972

(22) PCT Filed: May 26, 2011
(86) PCT No.: PCT/US11/38059

§ 371 Date: Mar. 27, 2013

Related U.S. Application Data
(60) Provisional application No. 61/349,038, filed on May 27, 2010.

(51) Int. Cl. A61B 3/12

(52) U.S. Cl. CPC A61B 3/12
USPC 351/215; 29/592; 29/428

(57) ABSTRACT

Various embodiments include systems and methods for ocular fundus examination reflection reduction. An illumination polarizer assembly can be configured to be user-attachable to an ophthalmological device and can be configured to position a first optical polarizer in an illumination portion of a light path associated with the ophthalmological device. An observation polarizer assembly can be configured to be user-attachable to the ophthalmological device and can also configured to position a second optical polarizer in an observation portion of the light path.

### CLAIM OF PRIORITY

This patent application claims the benefit of priority, under 35 U.S.C. Section 119(e), to U.S. Provisional Patent Application Ser. No. 61/349,038, entitled “Reflection Reduction System for Fundus Examination, Photography and Related Method,” filed on May 27, 2010, which is hereby incorporated by reference herein in its entirety.

### TECHNICAL FIELD

This document pertains generally, but not by way of limitation, to reflection reduction for ocular fundus examination.

### BACKGROUND

Clinical examination of the ocular fundus (hereinafter “fundus”) has typically relied on using an ophthalmological device to project light into the eye and capture the reflection of that light from ocular surfaces via optical assemblies for a clinician to view. Generally available ophthalmological devices used by clinicians include ophthalmic slit lamps (e.g., a device including a binocular stereomicroscope and an illumination source to provide a slit beam of light) and binocular indirect ophthalmoscopes. Clinicians generally employ a handheld lens (e.g., a condensing lens) to augment the ophthalmological device when examining the fundus. Reflections of the light off of the handheld lens, or other surfaces (e.g., the cornea), can produce visual artifacts impairing the clinician's view of the fundus. Example artifacts can include, for example, obscuring glare, reduction in image contrast, and effects resulting from pupil constriction of the clinician due to excessive reflected light. Clinicians generally manipulate the handheld lens, or adjust the light source (e.g., to narrow the slit beam) to reduce such reflections.

Some specialized ophthalmological devices have been constructed to use light polarization techniques for various purposes. For example, U.S. Pat. No. 4,998,818 mentions a direct ophthalmoscope with integrated polarizers to reduce glare. In another example, U.S. Pat. No. 4,711,541 mentions a slit lamp employing embedded polarizers, the slit lamp designed to record retroilluminated images of an eye's crystalline lens. In another example, U.S. Pat. No. 7,360,897, mentions integral polarizers as part of a device that estimates nerve fiber thickness by observing the light polarization properties of the nerve fibers. Other techniques have relied on software systems to remove aberrant reflections from captured images

### OVERVIEW

The present inventors have recognized, among other things, that manipulating the location or angle of a handheld lens to reduce aberrant reflections can reduce the viewable area of the fundus, hindering examination. Similarly, narrowing a slit beam of a slit lamp can also reduce the viewable area of the fundus. Narrowing the viewable area can result in increased time to examine the fundus as well as increased difficulty in performing procedures such as laser ablation of the peripheral retina to preserve central foveal vision.

The present inventors have also recognized that requiring replacement of existing devices can lead to increased costs for clinicians (and those who supply them), or may be impractical in many clinical settings. For example, clinicians, hospitals, or others would incur significant costs in entirely replacing the otherwise functional ophthalmological devices with newer devices having specialized integrated features. Further, techniques that merely rely on digital image processing can have limitations, such as again incurring unacceptable costs in terms of energy consumption, space, or computational complexity.

The present inventors have developed techniques and apparatus, such as including assemblies that can be user-attachable to retro-fit existing generally-available ophthalmological devices. Such user-attachable assemblies can provide reduction of aberrant reflection. In an example, a system of at least two optically coupled polarizer assemblies can be respectively attached to an ophthalmological device by a user to modify a light path associated with the ophthalmological device.

The light path associated with the ophthalmological device is the path of light that traverses from the ophthalmological device's light source (e.g., a lamp or other illumination source) to reflection locations (e.g., a handheld lens, the cornea, the fundus, etc.), and then to an observation location (e.g., an eye piece of a binocular stereomicroscope). The light path can be divided into two portions: an illumination portion and an observation portion. The illumination portion can be the portion of the light path, such as originating at an illumination source and terminating at the first reflection location (e.g., the first reflective surface encountered by the light path, such as a partially reflective mirror or splitter). The observation portion can be the second portion of the light path, such as originating at the first reflection location and terminating at a location of observation. A first polarizer assembly can be positioned by the user in the illumination portion of the light path such that an optical polarizer (e.g., a polarizing filter) secured by the first polarizer assembly transmits light having a first polarization axis. A second polarizer assembly can be positioned by the user in the observation portion of the light path such that an optical polarizer included as a portion of the second polarizer assembly transmits reflected light having a second polarization axis. In one example, the second polarization axis is perpendicular to the first polarization axis. Such cross polarization can reduce or eliminate reflections from surfaces that preferentially generate reflections having the first polarization axis (e.g., from highly reflective, or non-diffusing, surfaces).

In one example, one or more of the polarizer assemblies can be user-adjustable or user-manipulable to modify the polarization effects on the light path while remaining attached to the ophthalmological device. For example, one or more of the polarizer assemblies can be configured to flip, rotate, or otherwise move a secured optical polarizer into or out of the light path. In one example, a user can manipulate one or more of the polarizer assemblies to adjust the angle between the first polarization axis and the second polarization axis (e.g., to select or provide a desired observed degree of polarization selectivity or cross polarization, depending on clinical need). Providing these user adjustments can allow greater clinical utility of ophthalmological devices to users, including in the field (e.g., in a clinician's office). Examples of the increased ophthalmological device utility can include avoiding the light losses induced by the polarizers (e.g., by removing or adjusting one or more of the polarizer assemblies), as well as observing aspects of the fundus that appear different under varying polarization axis angles between the first polarization axis and the second polarization axis.

User-attachable, user-manipulable, or user-adjustable polarizer assemblies can provide the clinical benefits of using polarized light in fundus examinations (e.g., to reduce aberrant reflections) by retrofitting existing ophthalmologic examination equipment instead of requiring replacement of such equipment. The polarizer assemblies can provide a platform in which other optical devices (e.g., light spectrum filters) can be used to readily and cheaply augment existing ophthalmological devices. Thus, clinical flexibility of existing ophthalmological devices can be increased by a clinician resulting in increased efficiency and decreased costs.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example system for fundus examination reflection reduction, according to various embodiments.

FIG. 2 illustrates an example slit lamp ophthalmological device to which various embodiments can be applied.

FIG. 3 illustrates an example system for fundus examination reflection reduction applied to a slit lamp, according to various embodiments.

FIGS. 4A-D illustrate example mechanical supports for illumination and observation polarizer assemblies, according to various embodiments.

FIG. 5 illustrates an example indirect binocular ophthalmoscope ophthalmological device to which various embodiments can be applied.

FIGS. 6A-B illustrate different perspectives of an example system for fundus examination reflection reduction applied to an indirect binocular ophthalmoscope, according to various embodiments.

FIGS. 7A-B illustrate two perspectives of an example frame to secure and position illumination and observation polarizer assemblies, according to various embodiments.

FIG. 8 illustrates a flowchart of an example method to produce a system for fundus examination reflection reduction, according to various embodiments.

FIG. 9 illustrates a flowchart for an example method of fundus examination with reflection reduction, according to various embodiments.

FIGS. 10A-H and 11A-B illustrate general examples of retinal photography that can be obtained using apparatus and techniques such as those discussed below.

### DETAILED DESCRIPTION

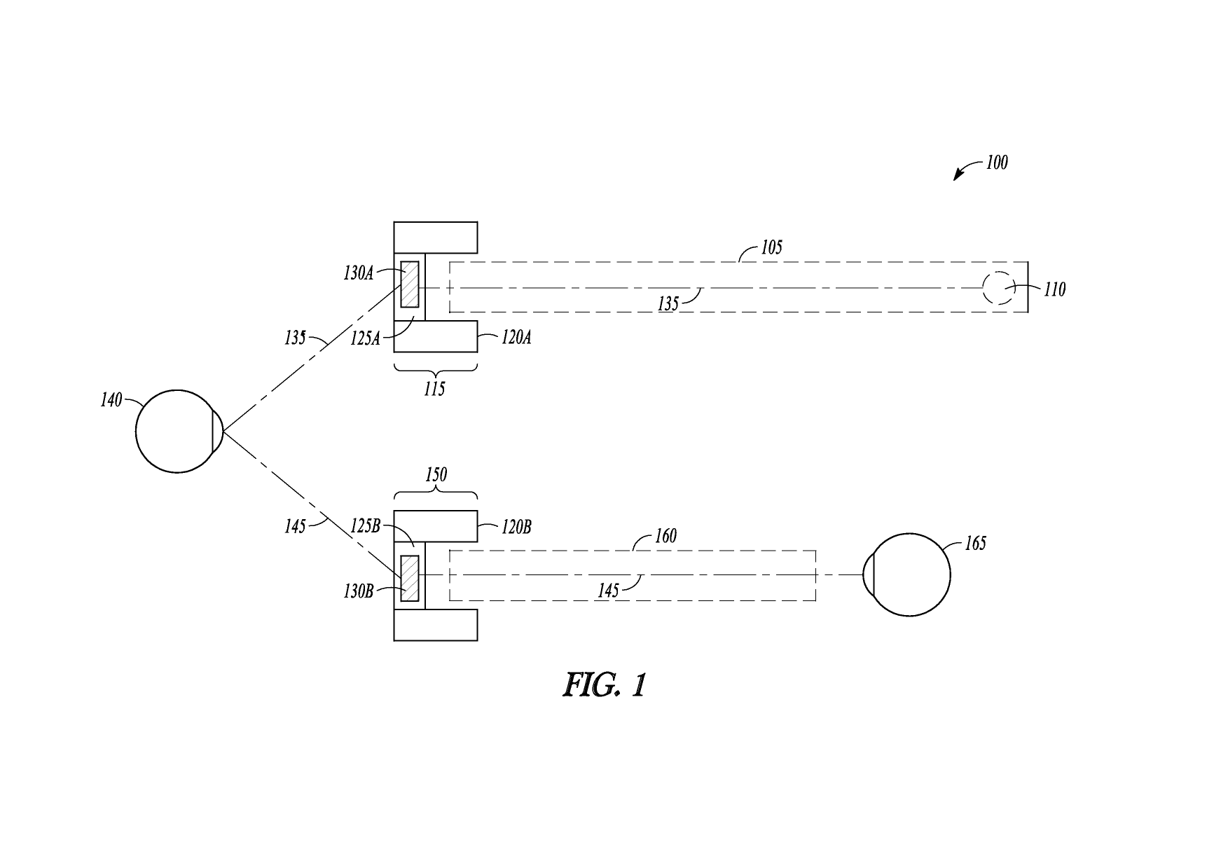

FIG. 1 illustrates an example system 100 for fundus examination reflection reduction. System 100 can include an illumination polarizer assembly 115 and an observation polarizer assembly 150.

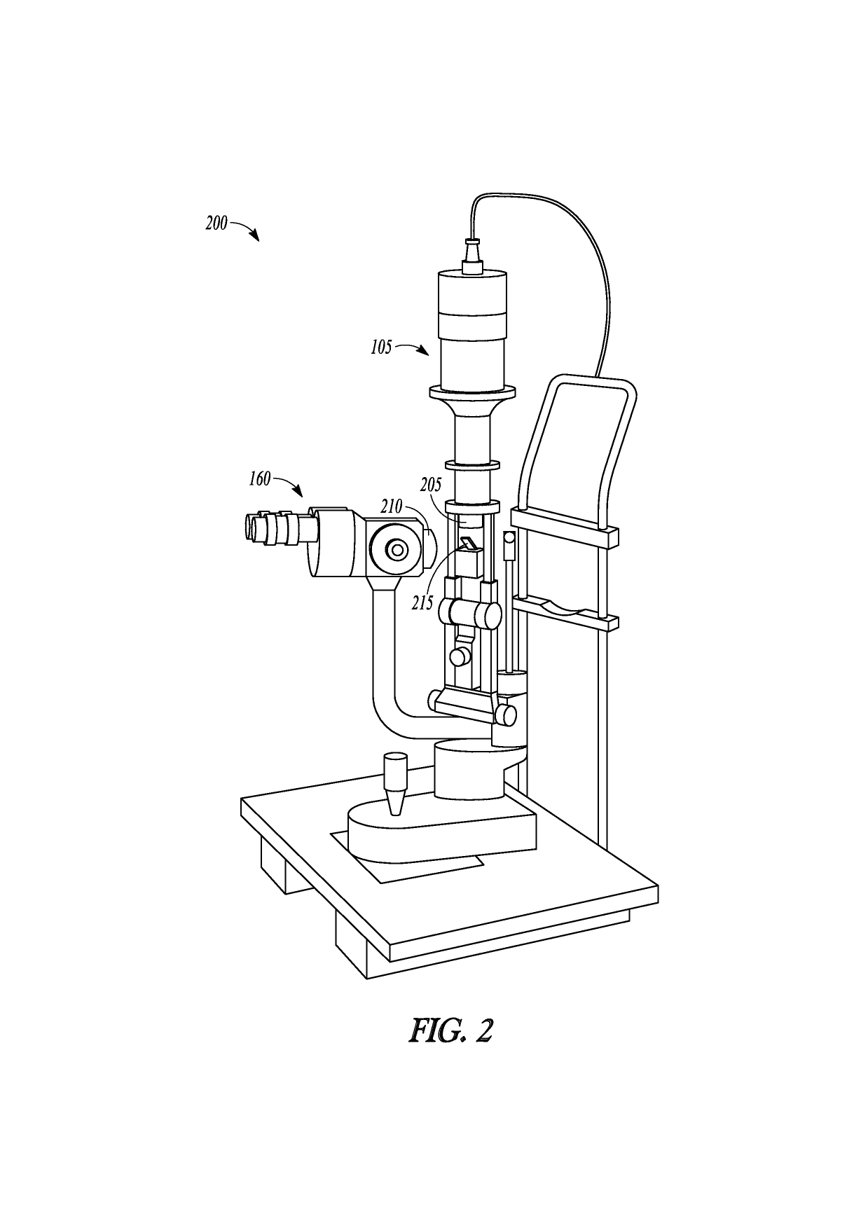

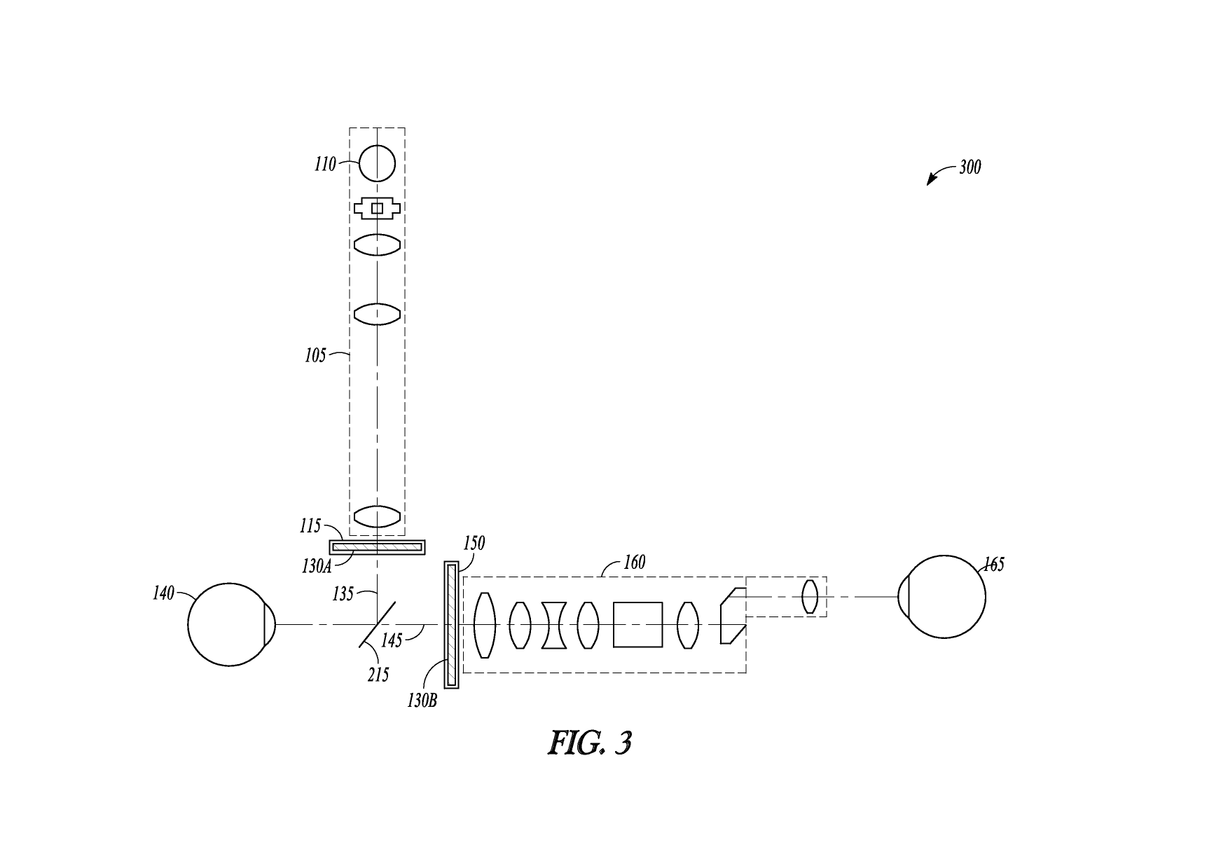

The illumination polarizer assembly 115 can be configured to be user-attachable to an ophthalmological device. The illumination polarizer assembly 115 can also be configured to position a first optical polarizer 130A in an illumination portion of the light path 135 associated with the ophthalmological device. For clarity, FIG. 1 includes a light source 110 that can be housed in an illumination module 105 of the ophthalmological device. In one example, the illumination portion of the light path 135 originates at the light source 110 location of the ophthalmological device and ends the reflective surface location 140. In one example, the reflective surface location 140 can be the first reflective surface encountered along the light path in a direction distal to the light source 110 location. In one example, the illumination portion of the light path 135 ends at a location where the illumination portion meets the observation portion of the light path 145 (e.g., a locus where the illumination portion occupies or traverses a similar spatial locus as the observation portion), such as discussed below in the example of FIGS. 2-3. In one example, additional light vectors can be demarcation locations for the illumination portion of the light path 135 or the observation portion of the light path 145. For example, when a laser is user with the ophthalmological device, for example in peripheral retina ablation, the illumination portion of the light path 135 or the observation portion of the light path 145 can be defined such as to avoid interfering with a path of the laser. In one example, the illumination portion of the light path 135 can terminate where it meets another light vector, such as a therapeutic laser used with, or integrated into the ophthalmological device.

The observation polarizer assembly 150 can be configured to be user-attachable to an ophthalmological device. The observation polarizer assembly 150 can be configured to position a second optical polarizer 130B in the observation portion of the light path 145. In one example, the observation portion of the light path 145 originates at the reflective surface location 140 and terminates at an observational location of the ophthalmological device (e.g., the observation module 160 or between the observation module 160 and the user 165), where the observational location can be configured for observation of an image of a target (e.g., a fundus region) by the user 165. In the example of FIG. 1, the user 165 is presented as a person, but other observational modes can be used, such as image capture via a digital or film camera, magnification and projection of an obtained image via optical or digital processing, or the like. In one example, the observation portion of the light path 145 begins at a location where the observation portion of the light path 145 meets the illumination portion of the light path illumination portion of the light path 135, as described above. In one example, the observation portion of the light path 145 begins where another light vector (e.g., from a laser) meets the light path as described above.

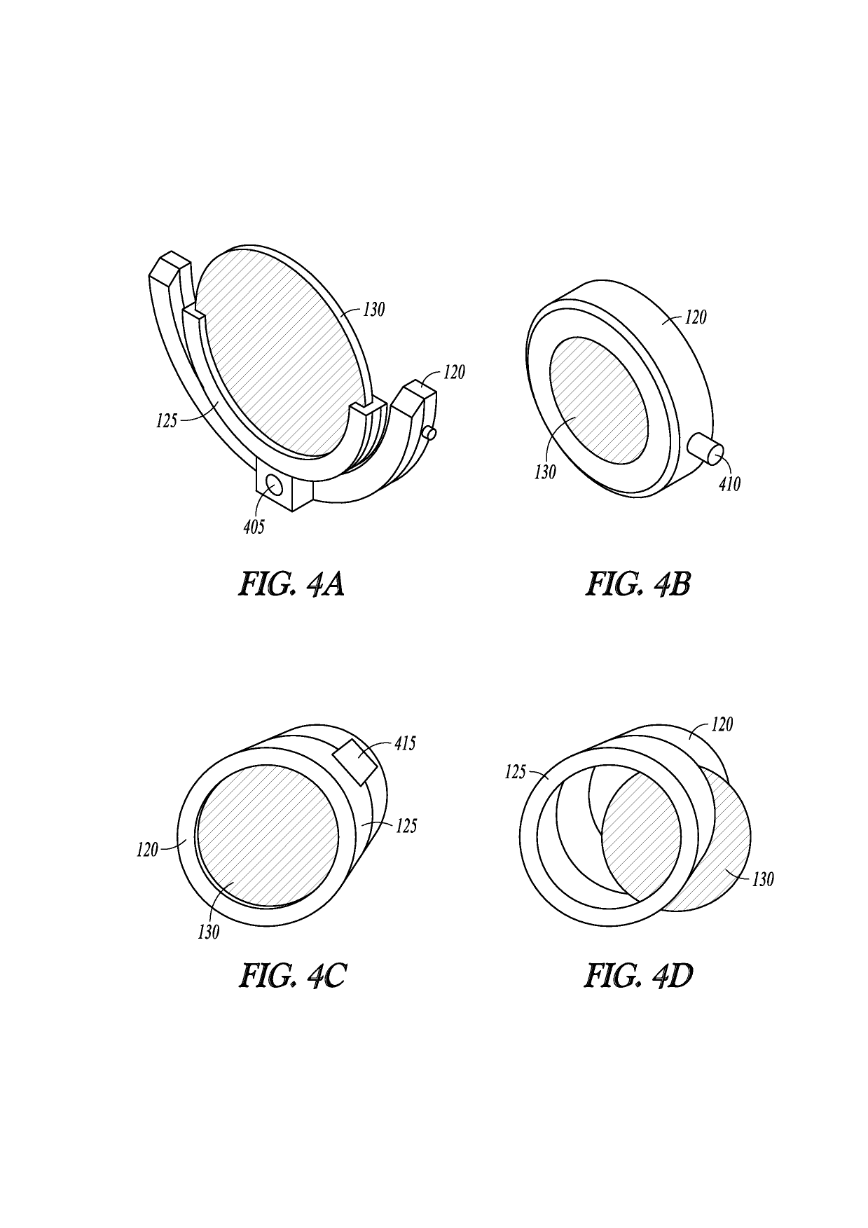

In one example, one or more of the illumination polarizer assembly 115 or the observation polarizer assembly 150 can comprise a respective mechanical support. In one example, mechanical support can include a respective mechanical coupler 120 (e.g., mechanical coupler 120a associated with the illumination polarizer assembly 115 or a mechanical coupler 120b associated with the observation polarizer assembly 150) configured to attach to an exterior surface of the ophthalmological device. In one example, the mechanical support can include a polarizer support 125 (e.g., polarizer support 125a associated with the illumination polarizer assembly 115 or polarizer support 125b associated with the observation polarizer assembly 150) coupled to the mechanical coupler 120 and configured to retain an optical polarizer 130 (e.g., a first optical polarizer 130A or a second optical polarizer 130B). One or more of the mechanical coupler 120 or the polarizer support 125 can be user-manipulable to position the optical polarizer 130 in relation to a respective illumination portion of the light path 135 or observation portion of the light path 145. Various configurations of the mechanical coupler 120 and the polarizer support 125 are described below in the examples of FIGS. 4A-D and 7A-B. The phrase “user-attachable” can refer to features of the mechanical support that permit a user to easily attach the illumination polarizer assembly 115 or the observation polarizer assembly 150 to the ophthalmological device, such as without the need for specialized tooling or training. For example, the mechanical support can be configured for attachment by hand by the user 165 (e.g., applying an adhesive, fastener, magnetic element, tightening a set-screw, actuating a press-fit, snap-on, or frictional coupling, etc.).

The phrases “user-manipulable” or “user-adjustable” can refer to features of the mechanical support that the user 165 can easily manipulate or easily adjust such as before or during the course of examination. For example, the mechanical support can be configured for hand adjustment by the user (e.g., via rotating the polarizer support 125). In one example, user 165 can use simple hand tools (e.g., a screw driver) to mount, manipulate, or adjust the mechanical support.

FIG. 2 illustrates an example slit lamp 200 ophthalmological device to which various examples can be attached or used. As shown in FIG. 2, the slit lamp 200 includes an illumination module 105 and an observation module 160. FIG. 2 includes an example of an illumination module attachment point 205, such as for the illumination polarizer assembly 115, and an example of an observation module attachment point 210, such as for the observation polarizer assembly observation polarizer assembly 150. This slit lamp 200 generally includes a mirror 215 to direct the illumination portion of the light path 135 into the target (e.g., the human eye).

FIG. 3 illustrates an example system 300 for reflection reduction applied to a slit lamp, such as for use during fundus examination. Specifically, system 300 illustrates an optical system diagram, such as including the examples of FIG. 1 as they would be arranged in relation to an example of the slit lamp 200. As mentioned above with respect to FIG. 1, system 300 illustrates generally the convergence of the illumination portion of the light path 135 and the observation portion of the light path 145 proximal to reflecting off of an exterior surface (e.g., reflective surface location 140 such as a cornea, or a handheld lens). For example, the mirror 215 redirects the light path so as to cause the illumination portion of the light path 135 and the observation portion of the light path 145 to converge and thus marks the termination of the illumination portion of the light path 135 and the origin of the observation portion of the light path 145.

FIGS. 4A-D illustrate generally examples of mechanical supports that can comprise a portion of the illumination 115 or observation 150 polarizer assemblies. In one example, the mechanical support can be configured to be user-manipulable to rotate the optical polarizer 130 about an axis parallel to a plane of the optical polarizer 130 (e.g., “flipping” the optical polarizer 130 on a hinge or pivot away from the mechanical coupler 120). In one example, one or more of the mechanical coupler 120 or the polarizer support 125 are user-manipulable to remove the optical polarizer 130 from the light path (e.g., flipping or sliding the optical polarizer 130 out of the light path).

In one example, the mechanical support can be configured to be user-manipulable to rotate the optical polarizer 130 about an axis perpendicular to a plane of the optical polarizer (e.g., the light path). In one example, the perpendicular axis is centered in the optical polarizer 130 and the polarization axis of light transmitted through the optical polarizer 130 is user-adjustable via rotation of the optical polarizer 130.

In one example, mechanical supports associated with one or more of the illumination polarizer assembly 115 or the observation polarizer assembly 150 can include a respective polarization orientation indicator configured to provide information indicative of the polarization orientation of a respective optical polarizer 130 (e.g., the first optical polarizer 130A or the second optical polarizer 130B). For example, the polarization orientation indicator can be configured to provide visual or tactile feedback indicative of the polarization orientation to the user 165. In one example, visual feedback is external to the mechanical support (e.g., visible markings can be included on the housing of the mechanical support or elsewhere). In one example, the visual feedback is provided internally and is visible, for example, to the user 165 when looking into or through the observation module 160.

FIG. 4A illustrates a mechanical support that can include a mechanical coupler 120 comprising a ring. The ring can be configured to encircle a portion of the exterior surface (e.g., illumination module attachment point 205 or observation module attachment point 205) of the ophthalmological device and anchor, for example, the illumination polarizer assembly 115 or the observation polarizer assembly 150 to the ophthalmological device. In one example, the ring can include an adhesive (e.g., a permanent or releasable adhesive, tape, etc.). In one example, the ring can include a magnetic element. In one example, the ring can include mounting holes for a strap, or can otherwise include a strap. In one example, other user applicable adhesion techniques, such as various fasteners (e.g., a fabric hook-and-loop fastener), can also be employed to attach the mechanical coupler 120 to the ophthalmological device. In one example, the attachment can be reversible, such as the mechanical support can be easily removed without damaging the ophthalmological device; for example, using a magnetic element.

The techniques described above do not require a ring-shaped structure for secure attachment of the polarizer assembly to the ophthalmological device. For example, the mechanical coupler 120 can include a partial ring, a tab, a flange, or other surface upon which the attachment techniques or apparatus discussed above can be used to attach the mechanical coupler 120 to the ophthalmological device.

In one example, the ring can be configured to anchor for example, the illumination polarizer assembly 115 or the observation polarizer assembly 150 via a frictional coupling. For example, the ring can be a tacky sleeve, such as including rubber or another elastic or deformable material, such as a material that can be stretched around an exterior portion of the ophthalmological device or compressed to provide an interference fit.

In one example, the mechanical support can be configured to be user-manipulable to rotate the optical polarizer 130 about an axis parallel to the plane of the optical polarizer 130. For example, user 165 can manually “flip” the polarizer support 125 using hinge 405, such as using one or more of the user's fingers, such as to remove the optical polarizer 130 from the light path. In one example, a pivot can rotate the optical polarizer such that a thin, non-transmissive, edge is within the light path (e.g., the plane of the optical polarizer is parallel with the light path but the optical polarizer remains within the light path). In this example, the optical polarizer is effectively removed from the light path (e.g., it does not polarize the light) but occupies the same cross sectional area or volume. Such volumetric compactness of the polarizer assembly allows the polarizer to be retrofit or installed on ophthalmologic devices where the available volume is relatively limited (e.g., in locations where a sliding or flipping configuration is impractical).

FIG. 4B illustrates a mechanical coupler 120 integrated with the polarizer support polarizer support 125 (not shown), including a set screw 410. The set screw 410 can be configured to immobilize the ring with respect to the ophthalmological device. In one example, the set screw 410 is configured to be tightened and loosened by the user's fingers. In one example, the set screw 410 is configured to be tightened and loosened by hand tools, such as a screw driver.

FIG. 4C illustrates a polarizer support 125 that rotationally slides against the mechanical coupler 120 to vary the polarization angle of the optical polarizer 130. In one example, an external feature of the polarizer support 125, such as a thumb tab 415, can be included to assist the user 165 in rotating the polarizer support 125. In one example, other features could also be employed, including rubberizing the external surface of the polarizer support 125 or texturizing the external surface via grooves etched into, or applied on top of, the polarizer support 125 housing.

FIG. 4D illustrates a polarizer support 125 including a frame configured to slidably position the optical polarizer 130. For example, the optical polarizer 130 can be slid into and out of the light path by the user 165 (such as shown in FIG. 4D, where the optical polarizer can be at least partially removed from the polarizer support 125). In one example, a handle, tab, button triggered spring, or other feature can be included in the polarizer support 125 to facilitate removal of the optical polarizer 130.

The various features described above with respect to FIGS. 4A-B can be combined in any way, such as increasing the user's ability to attach, manipulate, or otherwise adjust the polarization properties of the fundus examination reflection reduction system 100, while the mechanical support remains attached to the ophthalmological device. The present inventors have also recognized that the mechanical support (e.g., the polarizer support 125) can serve as a platform for securing additional optical components such as color filters, spatial filters, etc. In one example, the mechanical supports can include a separate optical component support (not shown) similar to the polarizer support 125.

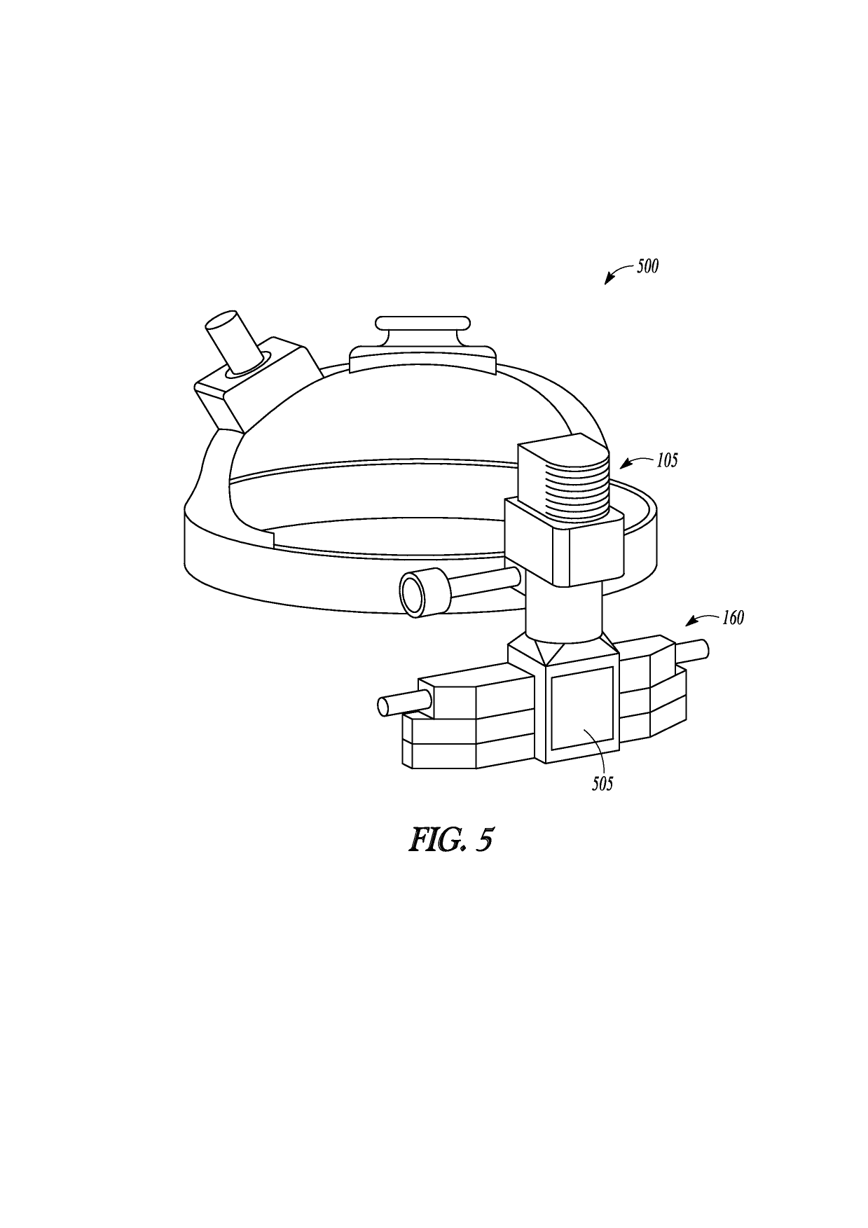

FIG. 5 illustrates an example indirect binocular ophthalmoscope 500 ophthalmological device to which various embodiments can be applied. As shown, the indirect binocular ophthalmoscope 500 can include an illumination module 105 and an observation module 160. In the example of FIG. 5, the light emission and receiving location 505 of the indirect binocular ophthalmoscope 500 is also shown. The light emission and receiving location 505 is the location from which the light path exits the indirect binocular ophthalmoscope 500 and light returning from the first reflection surface 140 (e.g., the fundus or hand-held lens) is redirected into the observation module is redirected into the observation module 160. In one example, coupling of the illumination 135 and observation 145 portions of the light path can include using a mirror and a stereo mirror assembly, the mirror placed such that the mirror does not intrude into the observation portion of the light path 145.

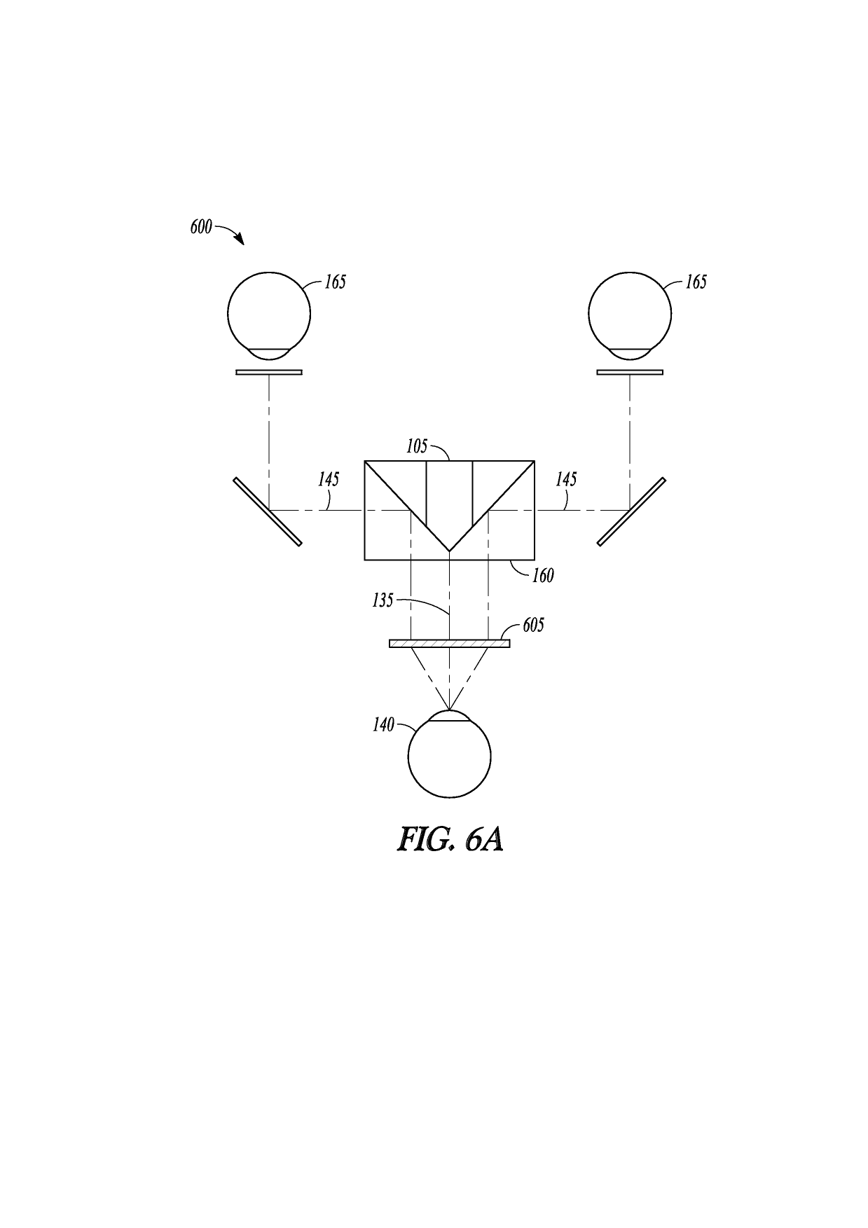

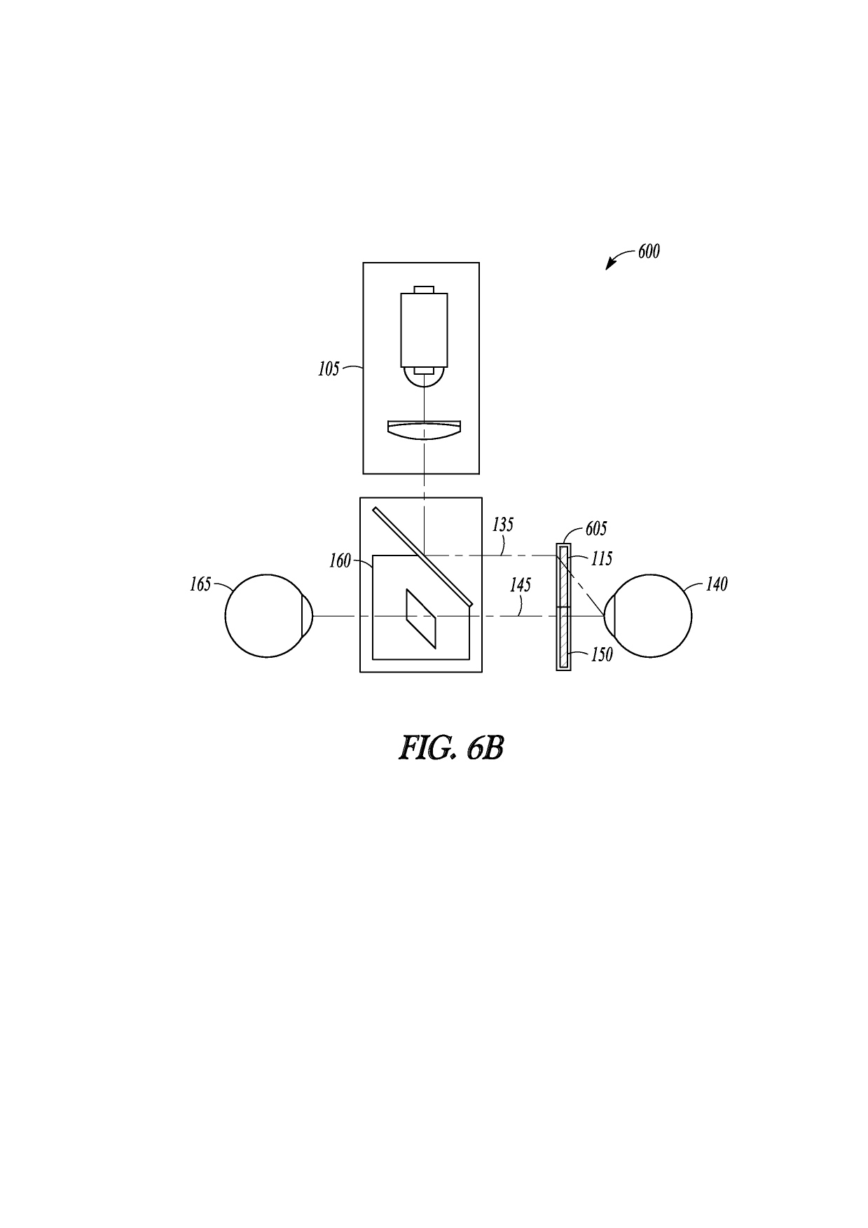

FIGS. 6A-B illustrate different perspectives of an example system 600 for fundus examination reflection reduction applied to an indirect binocular ophthalmoscope 500, according to various embodiments.

FIG. 6A illustrates a top-view of the system 600. System 600 can include an illumination module 105, and an observation module 160, and a corresponding illumination portion of the light path 135, and observation portion of the light path 145. The system 600 can include a frame 605 configured to secure the illumination polarizer assembly 115 and the observation polarizer assembly 150 near each other in about the same plane, such as shown in FIG. 6B. Frame 605 can be configured to orient the illumination polarizer assembly 115 to transmit light having a first polarization axis perpendicular to a second polarization axis and orient the observation polarizer assembly 150 to transmit light in the second polarization axis, reducing aberrant reflections off highly reflective (e.g., specular reflecting) surfaces.

The illumination polarizer assembly 115 can be held by the frame 605 above the observation polarizer assembly 150, as shown in FIG. 6B, such as because the illumination portion of the light path 135 of system 600 is horizontally superior to the observation portion of the light path 145. Other arrangements of the illumination polarizer assembly 115 and the observation polarizer assembly 150 can be used (e.g., arranged side-by-side, or in such a way as to occupy quadrants of the space within the frame 605).

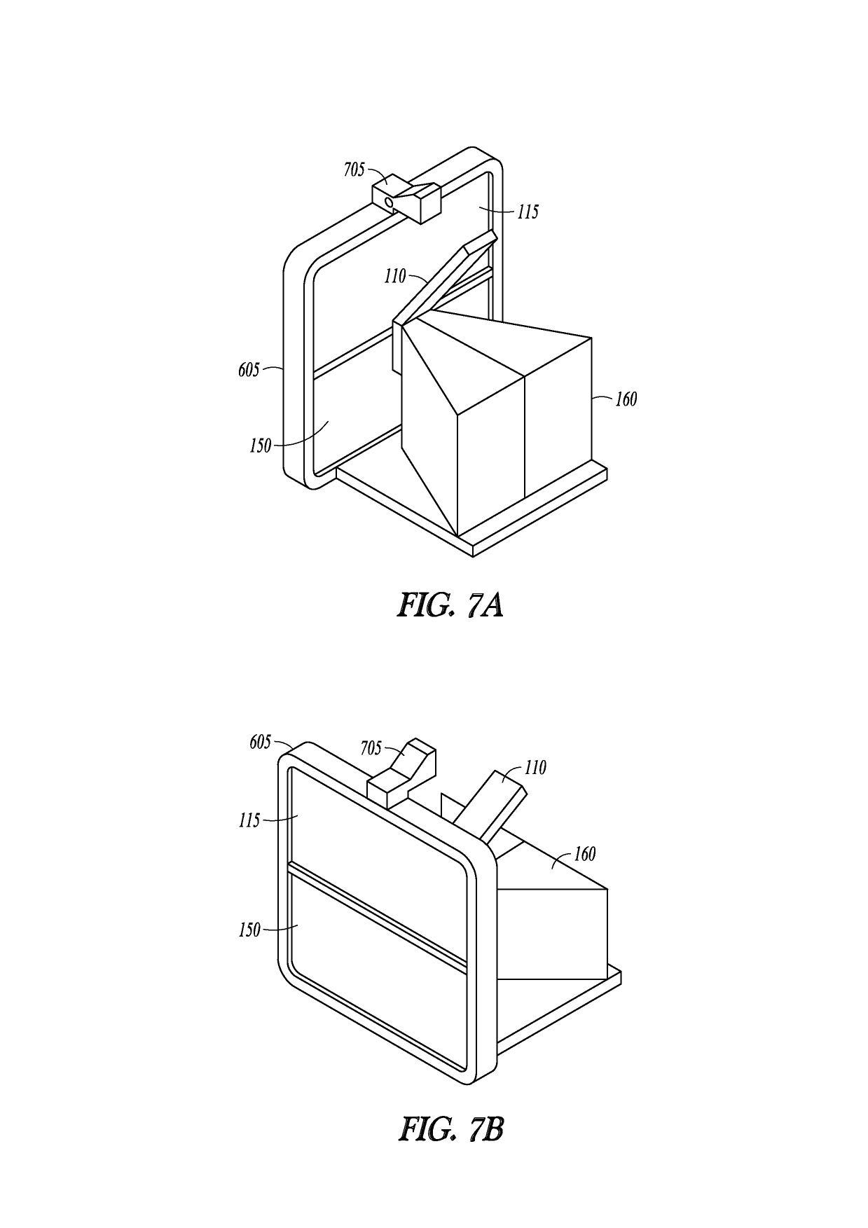

FIGS. 7A-B illustrate generally views of an example of a frame 605 that can be configured to secure or position illumination 115 and observation 150 polarizer assemblies.

FIG. 7A is a back-view, and FIG. 7B is a front view, of the frame 605. In one example, the frame 605 can include a hinge 705 that can be user-attached to the indirect binocular ophthalmoscope 500, via, for example, a strap, adhesive, magnetic element, or using one or more other attachment techniques. The hinge 705 can allow the user 165 to rotate (e.g., flip) the frame 605 out of the light path. In one example, the hinge 705 can be replaced with a swiveling arm, or a track upon which the frame 605 can slide laterally, such as to position the frame 605 within or out of the light path. In one example, the frame can be fastened to the hinge 705, such that it can snap on and off, or otherwise readily datable from the hinge 705.

As described above, existing ophthalmological devices can be retrofitted to achieve the clinical benefits of using polarized light. Further, the polarizer assemblies discussed above can be user-attachable, user-manipulable, or user-adjustable, to increase the utility of an existing ophthalmological device to the clinician. Examples include the ability to perform diagnostics tests with polarized and un-polarized light, or using polarizers to vary the angle between the first and the second polarization axes to suppress unwanted reflections or to highlight different features of the fundus that vary in visibility in relation to polarization conditions. By retrofitting existing equipment, clinicians can reduce costs while still achieving the desired clinical benefits described above.

Methods 800 and 900, described below, can include one or more components or techniques described above with respect to FIGS. 1-7. However, use of these components is not required to perform the operations of methods 800 and 900.

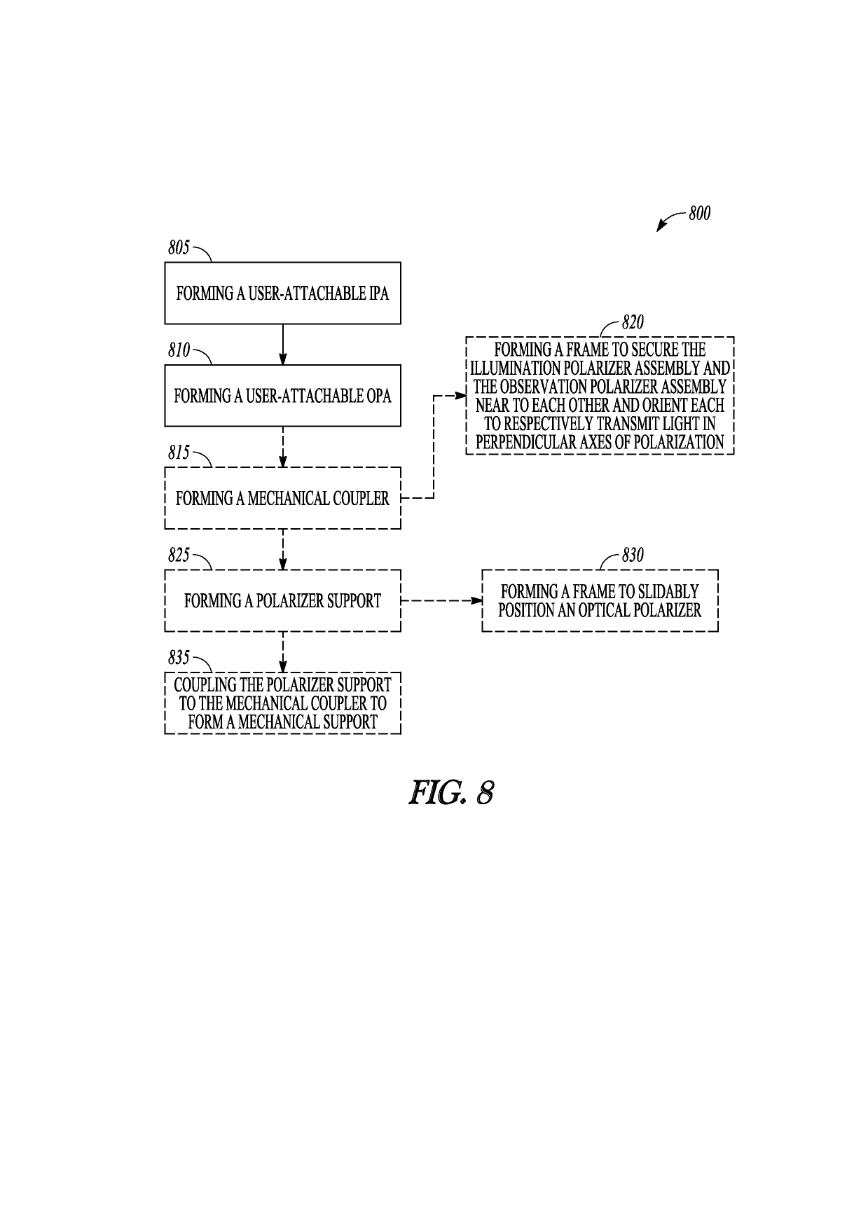

FIG. 8 illustrates generally a method 800 to provide a system for fundus examination reflection reduction, according to various embodiments.

At 805, an illumination polarizer assembly 115 can be formed. The illumination polarizer assembly 115 can be configured to be user-attachable to an ophthalmological device, such as those described above. The illumination polarizer assembly 115 can be configured to position a first optical polarizer 130A in an illumination portion 135 of a light path associated with the ophthalmological device.

At 810, an observation polarizer assembly 150 can be formed. The observation polarizer assembly 150 can be configured to be user-attachable to the ophthalmological device. The observation polarizer assembly 150 can be configured to position a second optical polarizer 130B in an observation portion of the light path 145. In one example, forming one or more of the illumination polarizer assembly 115 or the observation polarizer assembly 150 can include forming a mechanical support. In one example, the mechanical support can be formed to be user-manipulable to rotate the optical polarizer 130 about an axis parallel to a plane of the optical polarizer 130. In one example, the mechanical support can be formed to be user-manipulable to rotate the optical polarizer 130 about an axis perpendicular to a plane of the optical polarizer 130. In one example, the perpendicular axis can be centered in the optical polarizer 130 and a polarization axis of light transmitted through the optical polarizer 130 can be user-adjustable via rotation of the optical polarizer 130.

At operation 815, forming the mechanical support can include forming a mechanical coupler 120. The mechanical coupler 120 can be configured to attach to an exterior surface of the ophthalmological device. In one example, the mechanical coupler 120 can be formed to be user-manipulable to position the first optical polarizer 130A with respect to the illumination portion of the light path 135. In one example, forming the mechanical coupler 120 can include a ring that can be configured to encircle a portion of the exterior surface of the ophthalmological device. The ring can be configured to anchor the respective polarizer assembly (e.g., the illumination polarizer assembly 115 or the observation polarizer assembly 150) to the ophthalmological device.

At 820, forming the mechanical coupler 120 can include forming a frame 605. The frame 605 can be configured to secure the illumination polarizer assembly 115 and the observation polarizer assembly 150 near to each other in about the same plane. The frame can be configured to orient the illumination polarizer assembly 115 to transmit light having a first polarization axis perpendicular to a second polarization axis and orient the observation polarizer assembly 150 to transmit light having the second polarization axis.

At 825, forming the mechanical support can include forming a polarizer support 125. The polarizer support 125 can be configured to retain (e.g., hold or secure) an optical polarizer 130. In one example, the polarizer support 125 can be formed to be user-manipulable to position the second optical polarizer 130B with respect to the observation portion of the light path 145 (e.g., in or out of the observation portion of the light path 145).

At 830, forming the observation polarizer assembly 150 can include forming a frame configured to slidably position the optical polarizer optical polarizer 130, for example as shown in FIG. 4D.

At 835, the polarizer support 125 can be coupled (e.g., attached via hinge, interlocking flanges, etc.) to the mechanical coupler 120.

In one example, one or more of the mechanical coupler 120 or the polarizer support 125 are formed to be user-manipulable to remove the optical polarizer 130 from the light path. In one example, forming one or more of the mechanical coupler 120 or the polarizer support 125 can include a respective polarization orientation indicator configured to provide information indicative of the polarization orientation of a respective optical polarizer 130. In one example, the polarization indicator can be configured to provide visual or tactile feedback indicative of the polarization orientation to the user 165, for example, as described above with respect to FIGS. 4A-D.

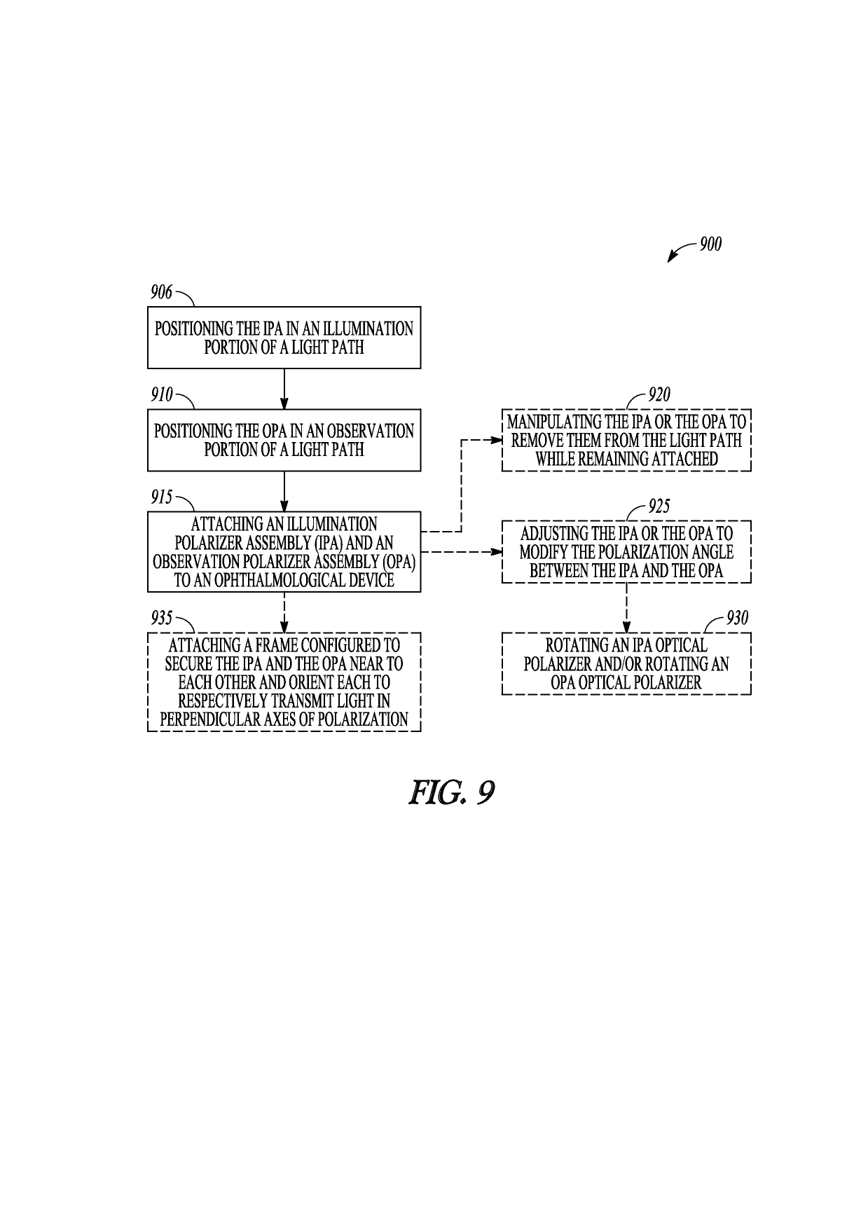

FIG. 9 illustrates a flowchart for an example method 900, performed by the user 165, of fundus examination with reflection reduction, according to various embodiments.

At 905, the user 165 positions the illumination polarizer assembly 115 in the illumination portion of the light path 135.

At 910, the user 165 positions the observation polarizer assembly 150 in the observation portion of the light path 145.

At 915, the user 165 can attach the illumination polarizer assembly 115 and the observation polarizer assembly 150 to the ophthalmological device. The illumination polarizer assembly 115 can include a first optical polarizer 130A and can be configured to transmit light (e.g., via the first optical polarizer 130A) having a first polarization axis. The observation polarizer assembly 150 can include a second optical polarizer 130B and can be configured to transmit light (e.g., via the second optical polarizer 130B) having a second polarization axis.

At 920, user 165 can manipulate one or more of the illumination polarizer assembly 115 or the observation polarizer assembly 150 to respectively remove the first optical polarizer 130A or the second optical polarizer 130B respectively from the illumination portion 135 or the observation portion 145 of the light path while the illumination polarizer assembly 115 and the observation polarizer assembly 145 remain attached the ophthalmological device.

At 925, user 165 can adjust one or more of the first optical polarizer 130A or the second optical polarizer 130B to modify the angle between the first polarization axis and the second polarization axis (e.g., to vary the angle between 0° and 90°). In one example, the user 165 can adjust one or more of the first optical polarizer 130A or the second optical polarizer 130B by rotating one or more of the first optical polarizer 130A or the second optical polarizer 130B until the second polarization axis is perpendicular to the first polarization axis.

At 935, user 165 can optionally attach a frame 605 to the exterior of the ophthalmological device. The frame 605 can be configured to secure the illumination polarizer assembly 115 and the observation polarizer assembly 150 near to each other in about the same plane. The frame 605 can be configured to orient the illumination polarizer assembly 115 or the observation polarizer assembly 150 to transmit light having the first polarization axis perpendicular to the second polarization axis.

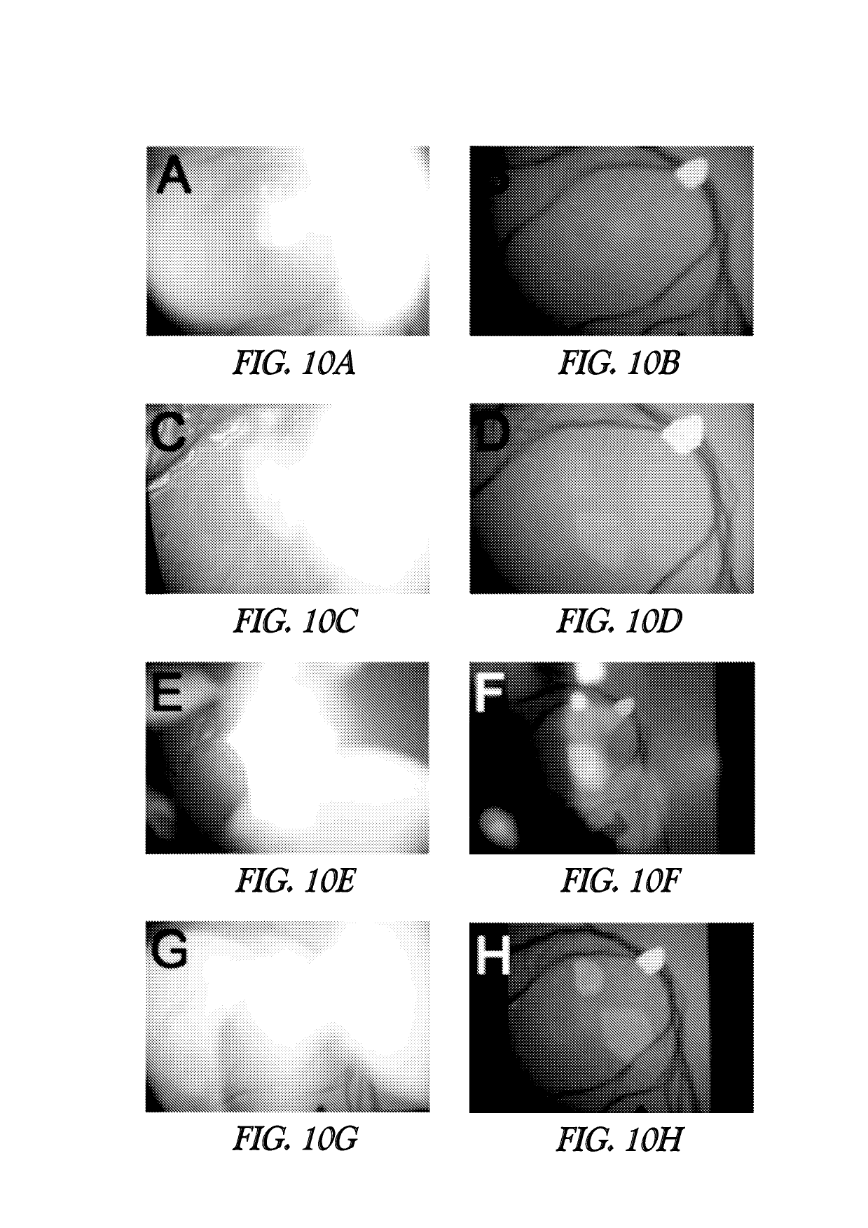

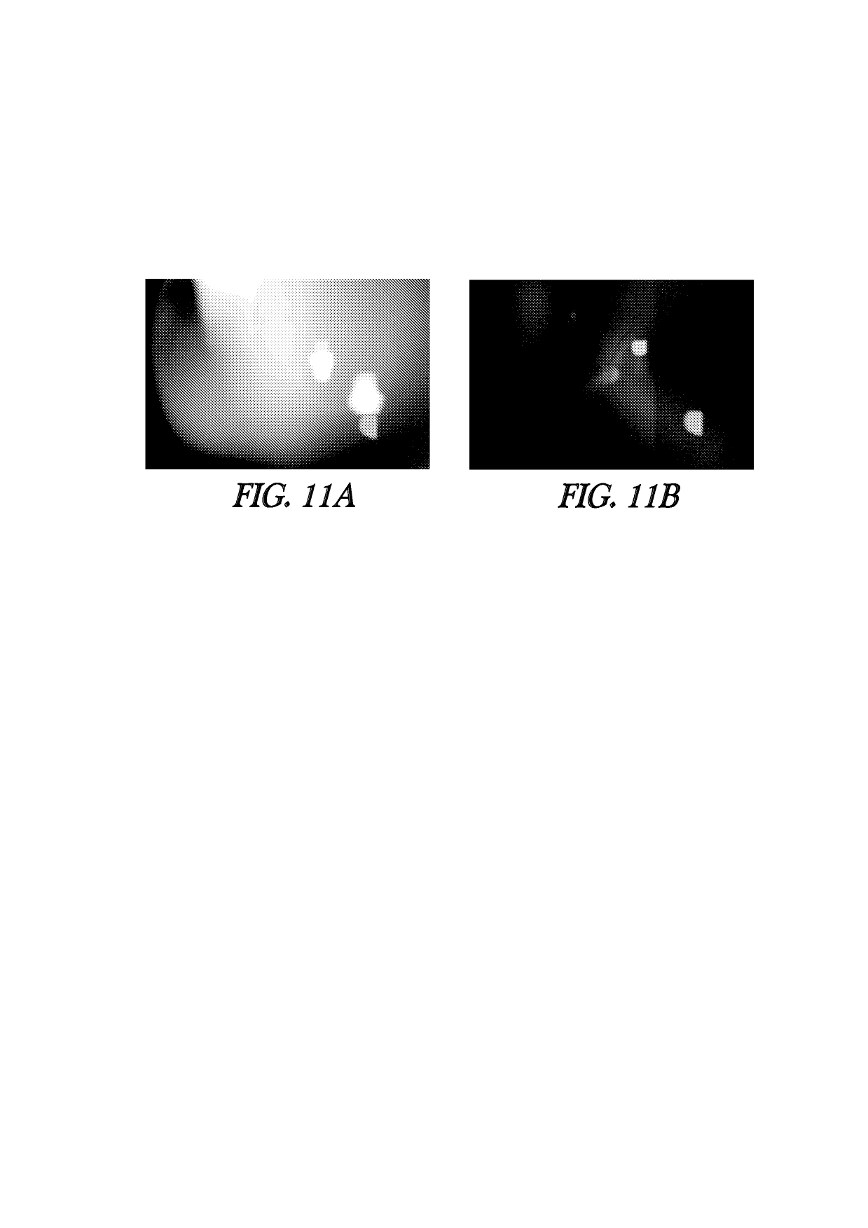

FIGS. 10A-H and 11 include generally illustrative examples of retinal photography that can be obtained using apparatus and techniques such as those discussed in the examples above. For example, FIGS. 10A, 10C, 10E, and 10G illustrate aberrant reflections from an illumination source. FIGS. 10B, 10D, 10F, and 10H respectively illustrate the images of FIGS. 10A, 10C, 10E, and 10G where the aberrant reflections have been suppressed using an illumination polarizer and an observation polarizer.

FIGS. 11A-B include images taken with a slit-lamp including an illumination polarizer assembly 115 and an observation polarizer assembly 150. FIG. 11A illustrates an example where the first polarization axis (e.g., the polarization axis associated with the illumination polarizer assembly 115) and the second polarization axis (e.g., the polarization axis associated with the observation polarizer assembly 150) are parallel. FIG. 11B illustrates an example where the first polarization axis and the second polarization axis are crossed (e.g., perpendicular) resulting in a marked decrease of aberrant reflections and improved fundus visualization.

### Additional Notes & Examples

Example 1 includes subject matter (such as a system) comprising an illumination polarizer assembly configured to be user-attachable to an ophthalmological device and configured to position a first optical polarizer in an illumination portion of a light path associated with the ophthalmological device, and an observation polarizer assembly configured to be user-attachable to the ophthalmological device and to position a second optical polarizer in an observation portion of the light path.

In Example 2, the subject matter of Example 1 can optionally include wherein one or more of the illumination polarizer assembly or the observation polarizer assembly comprises a respective mechanical support. The respective mechanical support can include a respective mechanical coupler configured to attach to an exterior surface of the ophthalmological device, a respective polarizer support coupled to the mechanical coupler and configured to retain an optical polarizer, and wherein one or more of the mechanical coupler or the polarizer support are user-manipulable to position the optical polarizer with respect to a respective illumination portion or observation portion of the light path.

In Example 3, the subject matter of Example 2 can optionally include wherein the polarizer support includes a frame configured to slidably position the optical polarizer.

In Example 4, the subject matter of one or any combination of Examples 2-3 can optionally include wherein the mechanical support is configured to be user-manipulable to rotate the optical polarizer about an axis parallel to a plane of the optical polarizer.

In Example 5, the subject matter of one or any combination of Examples 2-4 can optionally include wherein the mechanical support is configured to be user-manipulable to rotate the optical polarizer about an axis perpendicular to a plane of the optical polarizer.

In Example 6, the subject matter of Example 5 can optionally include wherein the axis is centered in the optical polarizer, and wherein a polarization axis of light transmitted through the optical polarizer is user-adjustable via rotation of the optical polarizer.

In Example 7, the subject matter of one or any combination of Examples 2-6 can optionally include wherein one or more of the mechanical coupler or the polarizer support are user-manipulable to remove the optical polarizer from the light path.

In Example 8, the subject matter of one or any combination of Examples 2-7 can optionally include wherein the mechanical coupler comprises a ring configured to encircle a portion of the exterior surface of the ophthalmological device, the ring configured to anchor the respective polarizer assembly to the ophthalmological device.

In Example 9, the subject matter of Example 8 can optionally include wherein the ring is configured to anchor the respective polarizer assembly to the ophthalmological device via a frictional coupling.

In Example 10, the subject matter of one or any combination of Examples 8-9 can optionally include wherein the ring includes a set screw configured to immobilize the ring with respect to the ophthalmological device.

In Example 11, the subject matter of one or any combination of Examples 1-10 can optionally include wherein the illumination portion of the light path originates at a light source location of the ophthalmological device and ends at a reflective surface location, the reflective surface location comprising the first reflective surface encountered along the light path in a direction distal to the light source location, and wherein the observation portion of the light path originates at the reflective surface location and terminates at an observational location of the ophthalmological device, the observational location configured for observation of an image of a target by the user.

In Example 12, the subject matter of one or any combination of Examples 1-11 can optionally include wherein one or more of the illumination polarizer assembly or the observation polarizer assembly includes a respective polarization orientation indicator configured to provide information indicative of the polarization orientation of a respective optical polarizer.

In Example 13, the subject matter of Example 12 can optionally include wherein the polarization orientation indicator is configured to provide visual or tactile feedback indicative of the polarization orientation to the user.

In Example 14, the subject matter of one or any combination of Examples 2-13 can optionally include wherein the mechanical coupler includes a frame. The frame can be configured to secure the illumination polarizer assembly and the observation polarizer assembly near each other in about the same plane, orient the illumination polarizer assembly to transmit light having a first polarization axis perpendicular to a second polarization axis, and orient the observation polarizer assembly to transmit light having the second polarization axis.

In Example 15, the subject matter of one or any combination of Examples 1-14 can optionally include wherein the ophthalmological device comprises one of a slit lamp or an indirect ophthalmoscope.

Example 16 can include, or can optionally be combined with the subject matter of one or any combination of Examples 1-15 to include, subject matter (such as a method, means for performing acts, or a machine-readable medium including instruction that, when performed by the machine cause the machine to perform acts) comprising forming an illumination polarizer assembly configured to be user-attachable to an ophthalmological device and configured to position a first optical polarizer in an illumination portion of a light path associated with the ophthalmological device, and forming an observation polarizer assembly configured to be user-attachable to the ophthalmological device and to position a second optical polarizer in an observation portion of the light path.

In Example 17, the subject matter of Example 16 can optionally include wherein forming one or more of the illumination polarizer assembly or the observation polarizer assembly comprises forming a mechanical support. The mechanical support can include forming a mechanical coupler configured to attach to an exterior surface of the ophthalmological device, forming polarizer support configured to retain an optical polarizer, coupling the polarizer support to the mechanical coupler, and wherein one or more of the mechanical coupler or the polarizer support are formed to be user-manipulable to position the optical polarizer with respect to a respective illumination portion or observation portion of the light path.

In Example 18, the subject matter of Example 17 can optionally include wherein forming the polarizer support includes forming a frame configured to slidably position the optical polarizer.

In Example 19, the subject matter of one or any combination of Examples 17-18 can optionally include wherein the mechanical support is formed to be user-manipulable to rotate the optical polarizer about an axis parallel to a plane of the optical polarizer.

In Example 20, the subject matter of one or any combination of Examples 17-19 can optionally include wherein the mechanical support is formed to be user-manipulable to rotate the optical polarizer about an axis perpendicular to a plane of the optical polarizer.

In Example 21, the subject matter of one or any combination of examples 17-20 can optionally include wherein the axis is centered in the optical polarizer, and wherein a polarization axis of light transmitted through the optical polarizer is user-adjustable via rotation of the optical polarizer.

In Example 22, the subject matter of one or any combination of Examples 17-21 can optionally include wherein one or more of the mechanical coupler or the polarizer support are formed to be user-manipulable to remove the optical polarizer from the light path.

In Example 23, the subject matter of one or any combination of Examples 17-22 can optionally include wherein forming the mechanical coupler includes a ring configured to encircle a portion of the exterior surface of the ophthalmological device, the ring configured to anchor the respective polarizer assembly to the ophthalmological device.

In Example 24, the subject matter of one or any combination of Examples 16-23 can optionally include wherein forming one or more of the mechanical coupler or the polarizer support includes a respective polarization orientation indicator configured to provide information indicative of the polarization orientation of a respective optical polarizer.

In Example 25, the subject matter of Example 24 can optionally include wherein the polarization indicator is configured to provide visual or tactile feedback indicative of the polarization orientation to the user.

In Example 26, the subject matter of one or any combination of Examples 17-25 can optionally include wherein forming the mechanical coupler includes forming a frame. The frame can be configured to secure the illumination polarizer assembly and the observation polarizer assembly near to each other in about the same plane, orient the illumination polarizer assembly to transmit light having a first polarization axis perpendicular to a second polarization axis, and orient the observation polarizer assembly to transmit light having the second polarization axis.

Example 27 can include, or can optionally be combined with the subject matter of one or any combination of Examples 1-26 to include, subject matter (such as a method, means for performing acts, or a machine-readable medium including instruction that, when performed by the machine cause the machine to perform acts) comprising attaching an illumination polarizer assembly and an observation polarizer assembly to an ophthalmological device, wherein the illumination polarizer assembly includes a first optical polarizer positioned in an illumination portion of a light path associated with the ophthalmological device and configured to transmit light having a first polarization axis, and

wherein the observation polarizer assembly includes a second optical polarizer positioned in an observation portion of the light path and configured to transmit light having a second polarization axis.

In Example 28, the subject matter of Example 27 can optionally include manipulating one or more of the illumination polarizer assembly or the observation polarizer assembly to respectively remove the first optical polarizer or the second optical polarizer respectively from the illumination portion or the observation portion of the light path, wherein the illumination polarizer assembly and the observation polarizer assembly remain attached the ophthalmological device.

In Example 29, the subject matter of one or any combination of Examples 27-28 can optionally include adjusting one or more of the first optical polarizer or the second optical polarizer to modify the angle between the first polarization axis and the second polarization axis.

In Example 30, the subject matter of Example 29 can optionally include wherein adjusting includes rotating one or more of the first optical polarizer or the second optical polarizer until the second polarization axis is perpendicular to the first polarization axis.

In Example 31, the subject matter of one or any combination of Examples 27-30 can optionally include wherein attaching the illumination polarizer assembly and the observation polarizer assembly to an exterior of an ophthalmological device includes attaching a frame to the exterior of the ophthalmological device. The frame can be configured to secure the illumination polarizer assembly and the observation polarizer assembly near to each other in about the same plane, orient the illumination polarizer assembly to transmit light having a first polarization axis perpendicular to a second polarization axis, and orient the observation polarizer assembly to transmit light having the second polarization axis.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document any documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A system comprising:
an illumination polarizer assembly configured to be user-attachable to an ophthalmological device and configured to position a first optical polarizer in an illumination portion of a light path associated with the ophthalmological device; and
an observation polarizer assembly configured to be user-attachable to the ophthalmological device and to position a second optical polarizer in an observation portion of the light path.

2. The system of claim 1, wherein one or more of the illumination polarizer assembly or the observation polarizer assembly comprises a respective mechanical support, the respective mechanical support including:
a respective mechanical coupler configured to attach to an exterior surface of the ophthalmological device;
a respective polarizer support coupled to the mechanical coupler and configured to retain an optical polarizer; and
wherein one or more of the mechanical coupler or the polarizer support are user-manipulable to position the optical polarizer with respect to a respective illumination portion or observation portion of the light path.

3. The system of claim 2, wherein the polarizer support includes a frame configured to slidably position the optical polarizer.

4. The system of claim 2, wherein the mechanical support is configured to be user-manipulable to rotate the optical polarizer about an axis parallel to a plane of the optical polarizer.

5. The system of claim 2, wherein the mechanical support is configured to be user-manipulable to rotate the optical polarizer about an axis perpendicular to a plane of the optical polarizer.

6. The system of claim 5, wherein the axis is centered in the optical polarizer; and
wherein a polarization axis of light transmitted through the optical polarizer is user-adjustable via rotation of the optical polarizer.

7. The system of claim 2, wherein one or more of the mechanical coupler or the polarizer support are user-manipulable to remove the optical polarizer from the light path.

8. The system of claim 2, wherein the mechanical coupler comprises a ring configured to encircle a portion of the exterior surface of the ophthalmological device, the ring configured to anchor the respective polarizer assembly to the ophthalmological device.

9. The system of claim 8, wherein the ring is configured to anchor the respective polarizer assembly to the ophthalmological device via a frictional coupling.

10. The system of claim 8, wherein the ring includes a set screw configured to immobilize the ring with respect to the ophthalmological device.

11. The system of claim 1, wherein the illumination portion of the light path originates at a light source location of the ophthalmological device and ends at a reflective surface location, the reflective surface location comprising the first reflective surface encountered along the light path in a direction distal to the light source location; and
wherein the observation portion of the light path originates at the reflective surface location and terminates at an observational location of the ophthalmological device, the observational location configured for observation of an image of a target by the user.

12. The system of claim 1, wherein one or more of the illumination polarizer assembly or the observation polarizer assembly includes a respective polarization orientation indicator configured to provide information indicative of the polarization orientation of a respective optical polarizer.

13. The system of claim 12, wherein the polarization orientation indicator is configured to provide visual or tactile feedback indicative of the polarization orientation to the user.

14. The system of claim 2, wherein the mechanical coupler includes a frame configured to:
secure the illumination polarizer assembly and the observation polarizer assembly near each other in about the same plane;
orient the illumination polarizer assembly to transmit light having a first polarization axis perpendicular to a second polarization axis; and
orient the observation polarizer assembly to transmit light having the second polarization axis.

15. The system of claim 1, wherein the ophthalmological device comprises one of a slit lamp or an indirect ophthalmoscope.

16. A method comprising:
forming an illumination polarizer assembly configured to be user-attachable to an ophthalmological device and configured to position a first optical polarizer in an illumination portion of a light path associated with the ophthalmological device; and
forming an observation polarizer assembly configured to be user-attachable to the ophthalmological device and to position a second optical polarizer in an observation portion of the light path.

17. The method of claim 16, wherein forming one or more of the illumination polarizer assembly or the observation polarizer assembly comprises forming a mechanical support including:
forming a mechanical coupler configured to attach to an exterior surface of the ophthalmological device;
forming polarizer support configured to retain an optical polarizer;
coupling the polarizer support to the mechanical coupler; and
wherein one or more of the mechanical coupler or the polarizer support are formed to be user-manipulable to position the optical polarizer with respect to a respective illumination portion or observation portion of the light path.

18. The method of claim 17, wherein forming the polarizer support includes forming a frame configured to slidably position the optical polarizer.

19. The method of claim 17, wherein the mechanical support is formed to be user-manipulable to rotate the optical polarizer about an axis parallel to a plane of the optical polarizer.

20. The method of claim 17, wherein the mechanical support is formed to be user-manipulable to rotate the optical polarizer about an axis perpendicular to a plane of the optical polarizer.

21. The method of claim 17, wherein the axis is centered in the optical polarizer; and
wherein a polarization axis of light transmitted through the optical polarizer is user-adjustable via rotation of the optical polarizer.

22. The method of claim 17, wherein one or more of the mechanical coupler or the polarizer support are formed to be user-manipulable to remove the optical polarizer from the light path.

23. The method of claim 17, wherein forming the mechanical coupler includes a ring configured to encircle a portion of the exterior surface of the ophthalmological device, the ring configured to anchor the respective polarizer assembly to the ophthalmological device.

24. The method of claim 16, wherein forming one or more of the mechanical coupler or the polarizer support includes a respective polarization orientation indicator configured to provide information indicative of the polarization orientation of a respective optical polarizer.

25. The method of claim 24, wherein the polarization indicator is configured to provide visual or tactile feedback indicative of the polarization orientation to the user.

26. The method of claim 17, wherein forming the mechanical coupler includes forming a frame configured to:
secure the illumination polarizer assembly and the observation polarizer assembly near to each other in about the same plane;
orient the illumination polarizer assembly to transmit light having a first polarization axis perpendicular to a second polarization axis; and
orient the observation polarizer assembly to transmit light having the second polarization axis.

27. A method performed by a user of an ophthalmological device, the method comprising:
attaching an illumination polarizer assembly and an observation polarizer assembly to an ophthalmological device;
wherein the illumination polarizer assembly includes a first optical polarizer positioned in an illumination portion of a light path associated with the ophthalmological device and configured to transmit light having a first polarization axis; and
wherein the observation polarizer assembly includes a second optical polarizer positioned in an observation portion of the light path and configured to transmit light having a second polarization axis.

28. The method of claim 27, comprising manipulating one or more of the illumination polarizer assembly or the observation polarizer assembly to respectively remove the first optical polarizer or the second optical polarizer respectively from the illumination portion or the observation portion of the light path, wherein the illumination polarizer assembly and the observation polarizer assembly remain attached the ophthalmological device.

29. The method of claim 27, comprising adjusting one or more of the first optical polarizer or the second optical polarizer to modify the angle between the first polarization axis and the second polarization axis.

30. The method of claim 29, wherein adjusting includes rotating one or more of the first optical polarizer or the second optical polarizer until the second polarization axis is perpendicular to the first polarization axis.

31. The method of claim 27, wherein attaching the illumination polarizer assembly and the observation polarizer assembly to an exterior of an ophthalmological device includes attaching a frame to the exterior of the ophthalmological device, the frame configured to:
secure the illumination polarizer assembly and the observation polarizer assembly near to each other in about the same plane;
orient the illumination polarizer assembly to transmit light having a first polarization axis perpendicular to a second polarization axis; and
orient the observation polarizer assembly to transmit light having the second polarization axis.
